(19) United States
(12) Patent Application Publication
(10) Pub. No.: US 2022/0212028 A1
(43) Pub. Date: Jul. 7, 2022

(54) LIGHT GUIDE TYPE LED MASK DEVICE

(71) Applicant: Softwells Co., Ltd., Gwangju (KR)

(72) Inventor: Seung Woong Lee, Gyeonggi-do (KR)

(21) Appl. No.: 17/603,852

(22) PCT Filed: Oct. 17, 2019
(86) PCT No.: PCT/KR2019/013683

§ 371 Date: Oct. 14, 2021

(30) Foreign Application Priority Data
Apr. 18, 2019 (KR) 10-2019-0045613

(51) Int. Cl. A61N 5/06

(52) U.S. Cl. CPC A61N 5/0616; A61N 2005/0652; A61N 2005/0647; A61N 2005/0662; A61N 2005/0666

(57) ABSTRACT

Disclosed is a light-guide-type LED mask device comprising a support unit having a front surface opening hole and a rear surface insertion hole for providing a path through which the face of the head and the front side of the head part are inserted, a light-emitting unit for emitting visible light or near-infrared rays and comprising a light-guide-type light-emitting module which is attached to the outer circumferential surface of the front surface opening hole, a light guide unit coupled to the front surface opening hole; and a reflective unit which contacts the front surface of the light guide unit and induces light leaking towards the front surface of the light guide unit into the light guide unit.

### TECHNICAL FIELD

The present disclosure relates to a light guide type LED mask device.

### BACKGROUND ART

Recently, as skin care using an LED light source radiating visible or near-infrared light has become a trend, products relating to the above are growing into a large market in a cosmetic industry and a skin care industry. Unlike a high-power laser, an LED light source may radiate an appropriate optical power to a large area of a disease site, and similar to the principle in which sunlight is converted into plant cells through chlorophyll in plants, a skin treatment technology using the LED light source promotes a basic energy metabolism of mitochondria in cells by radiating LED light and induces a photo-biochemical reaction between skin cells.

The cosmetics industry has released a variety of anti-aging-related cosmetics using LED light sources, and the skin care industry has provided various skin care services related to anti-aging. In relation to skin care devices, in recent, products that can manage skin or scalp more easily and conveniently using an LED element outputting a wavelength in a visible ray region or an LED element outputting a wavelength in a near infrared region have been released.

A conventional LED mask device relates to an optical mask device for skin care, this LEED mask device includes a face mask that may be mounted on a face of the human body, a light-emitting unit mounted in the face mask to emit light to a user's face wearing the face mask, a light emission driving part for turning on/off a driving of the light-emitting unit, a manipulation part for selecting driving of the light-emitting unit, and a control unit that controls the light emission driving part according to an operation signal of the manipulation part. In the above conventional LED mask device, a LED light source is arranged on a light-emitting unit on the whole, so that visible light or near-infrared light of the LED light source is directly radiated to a user's face. Therefore, as shown in FIG. 24, light radiated from the LED light source is radiated while forming a hot spot. In the region where light is radiated, 60 to 70% of light is concentrated on 10% of the unit area and 70 to 80% of light is concentrated on 30% of the unit area, so that the amount of light radiated to 10% of the region inside a hot spot and the amount of light radiated to 70% of the outside region are 14 times different, and as result, the amount of light radiated to a specific region is relatively large. Accordingly, there is a problem in that a relatively large amount of visible or near-infrared light is radiated to a skin in a region directly below the LED element and relatively little visible or near-infrared light is radiated to the skin in a region farther from the LED element.

### DISCLOSURE OF THE INVENTION

### Technical Problem

An object of the present disclosure is to provide a light-guiding type LED mask device which uniformly radiates LED light such as visible light or near infrared light to an entire face skin.

### Technical Solution

A light-guiding type LED mask device according to one embodiment of the present disclosure may include a support unit formed in a shape corresponding to an entire face and a portion of a front side of a head on the whole, and being provided with a front opening and a rear insertion opening providing a path into which the face and the front side of the head are inserted; a light-emitting unit configured to emit visible light or near-infrared light and provided with a light-guiding type light-emitting module attached to an outer periphery surface of the front opening; a light-guiding unit coupled to the front opening and radiating light radiated from the light-guiding type light-emitting module; and a reflection unit being in contact with a front surface of the light-guiding unit and configured to allow light, which travels to the front surface of the light-guiding unit, to enter the inside of the light-guiding unit.

### Advantageous Effects

The light-guiding type LED mask device of the present disclosure has an effect of uniformly radiating LED light such as visible light or near-infrared light to an entire skin of user's face.

### BRIEF DESCRIPTION OF THE DRAWINGS

FIG. 1 is a perspective view of a light-guiding type LED mask device according to one embodiment of the present disclosure.

FIG. 2 is an exploded perspective view of the light-guiding type LED mask device according to one embodiment of the present disclosure.

FIG. 3 is a rear view of the light-guiding type LED mask device according to one embodiment of the present disclosure.

FIG. 4 is a front view of a light-emitting unit of the light-guiding type LED mask device according to one embodiment of the present disclosure.

FIG. 5 is a perspective view of the light-emitting unit of the light-guiding type LED mask device according to one embodiment of the present disclosure.

FIG. 6 is a perspective view showing a state in which the light-emitting unit of the light-guiding type LED mask device according to one embodiment of the present disclosure is seated on a support unit.

FIG. 7 is a side view showing a state in which the light-emitting unit of the light-guiding type LED mask device according to one embodiment of the present disclosure is seated on a light-guiding unit.

FIG. 8 is a perspective view showing a state in which the light-emitting unit of the light-guiding type LED mask device according to one embodiment of the present disclosure is seated between the light-guiding unit and the support unit.

FIG. 9 is a partial vertical sectional view showing a coupling state of the light-emitting unit, the light-guiding unit and the support unit of the light-guiding type LED mask device according to one embodiment of the present disclosure.

FIG. 10 is a perspective view of a light-guiding type light-emitting module of the light-guiding type LED mask device according to one embodiment of the present disclosure.

FIG. 11 is a perspective view of a direct type light-emitting module of the light-guiding type LED mask device according to one embodiment of the present disclosure.

FIG. 12 is a partial vertical sectional view showing a coupling state of the light-emitting unit, the light-guiding unit, the support unit and a reflection unit of the light-guiding type LED mask device according to one embodiment of the present disclosure.

FIG. 13 is a partial vertical sectional view showing a coupling state of the light-emitting unit, the light-guiding unit, the support unit and the reflection unit of the light-guiding type LED mask device according to another embodiment of the present disclosure.

FIG. 14 is a partial vertical sectional view showing a coupling state of the light-emitting unit, the light-guiding unit and the reflection unit of the light-guiding type LED mask device according to still another embodiment of the present disclosure.

FIG. 15 is a partial vertical sectional view showing a coupling state of the light-emitting unit, the light-guiding unit and the reflection unit of the light-guiding type LED mask device according to still another embodiment of the present disclosure.

FIG. 16 is a partial vertical sectional view showing a coupling state of the light-emitting unit, the light-guiding unit and the reflection unit of the light-guiding type LED mask device according to still another embodiment of the present disclosure.

FIG. 17 is a schematic view illustrating a state in which light radiated from the light-emitting part of the light-guiding type LED mask device according to one embodiment is being dispersed by the light-guiding unit and the reflection unit.

FIG. 18 is a schematic configuration view for a control unit of the light-guiding type LED mask device according to one embodiment of the present disclosure.

FIG. 19 is a view showing a mask model, a LED distribution and a pattern form for calculating a light extraction degree distribution of the light-guiding type LED mask device according to one embodiment of the present disclosure.

FIG. 20 is a view showing a specific form of a light-guiding pattern used in FIG. 19.

FIG. 21 is a model picture illustrating locations at which the light extraction degree distribution is measured in the mask of FIG. 19.

FIG. 22 is a graph for the light extraction degree distribution evaluated in eight directions of FIG. 21.

FIG. 23 is a graph for the light extraction degree distribution evaluated in a state in which no light-guiding pattern is formed.

FIG. 24 is a graph for luminous intensity distribution evaluated in a state in which a contour-shaped light-guiding pattern is formed and no radial-shaped light-guiding pattern is formed.

FIG. 25 is a photograph showing a light radiation state of a conventional light-guiding type LED mask device.

### BEST MODE FOR CARRYING OUT THE INVENTION

Hereinafter, a light-guiding type LED mask device according to preferred embodiments of the present disclosure is described in detail with reference to the accompanying drawings.

Firstly, a structure of a light-guiding type LED mask device according to one embodiment of the present disclosure is described.

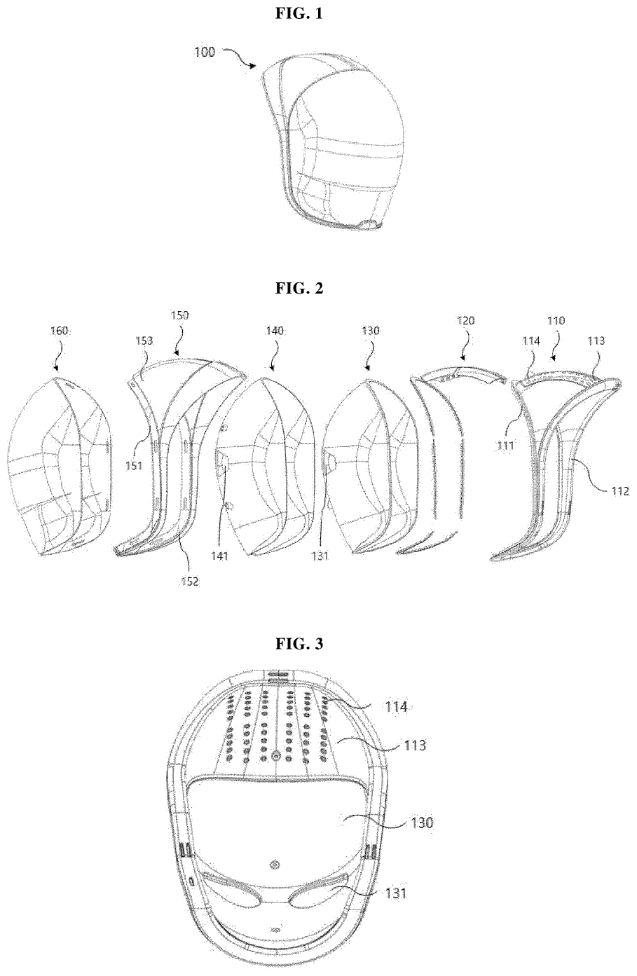

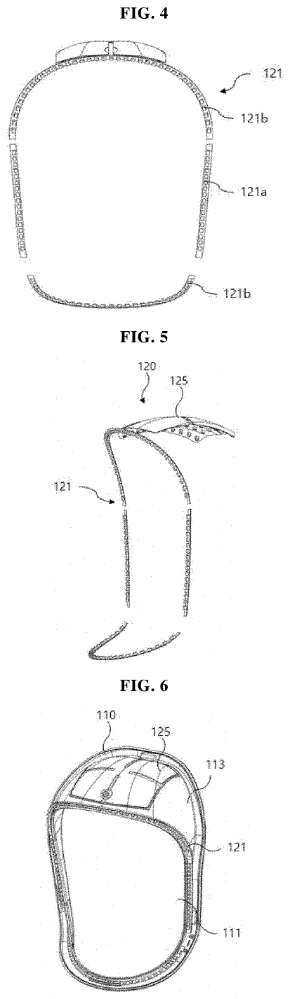

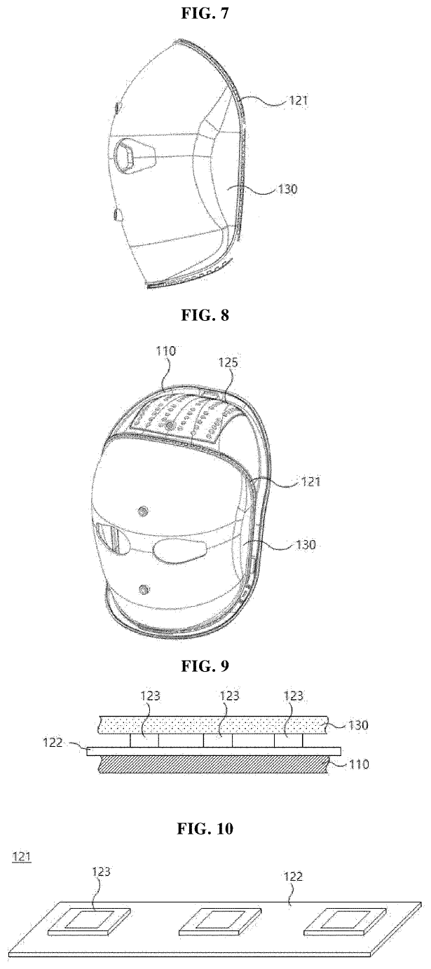

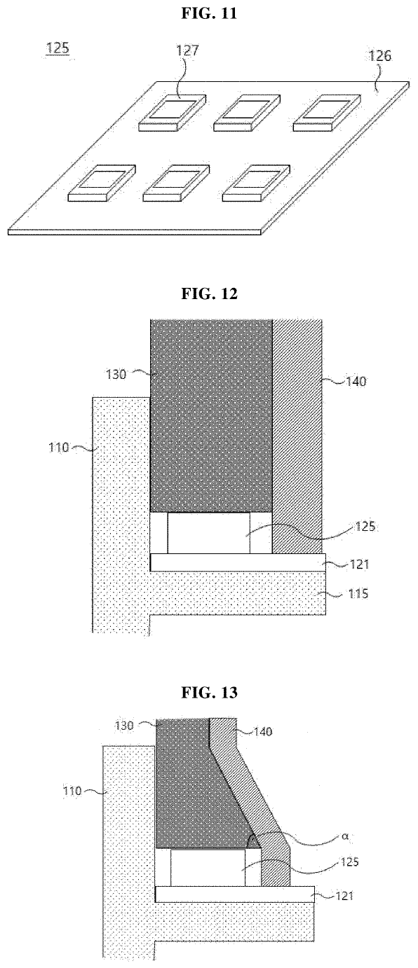

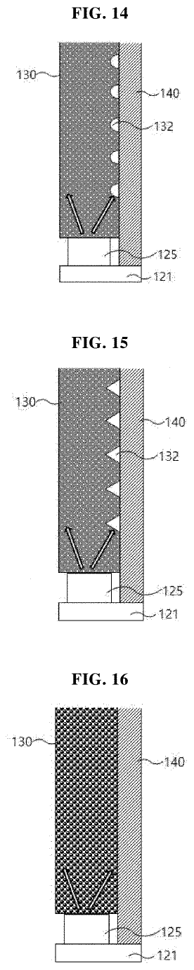

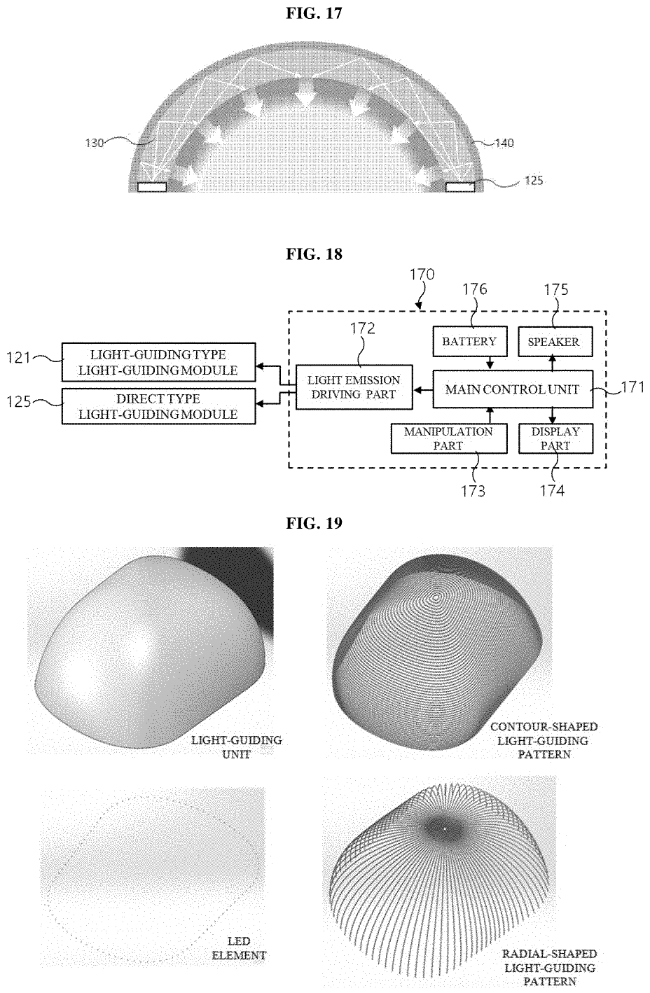

FIG. 1 is a perspective view of a light-guiding type LED mask device according to one embodiment of the present disclosure. FIG. 2 is an exploded perspective view of the light-guiding type LED mask device according to one embodiment of the present disclosure. FIG. 3 is a rear view of the light-guiding type LED mask device according to one embodiment of the present disclosure. FIG. 4 is a front view of a light-emitting unit of the light-guiding type LED mask device according to one embodiment of the present disclosure. FIG. 5 is a perspective view of the light-emitting unit of the light-guiding type LED mask device according to one embodiment of the present disclosure. FIG. 6 is a perspective view showing a state in which the light-emitting unit of the light-guiding type LED mask device according to one embodiment of the present disclosure is seated on a support unit. FIG. 7 is a side view showing a state in which the light-emitting unit of the light-guiding type LED mask device according to one embodiment of the present disclosure is seated on a light-guiding unit. FIG. 8 is a perspective view showing a state in which the light-emitting unit of the light-guiding type LED mask device according to one embodiment of the present disclosure is seated between the light-guiding unit and the support unit. FIG. 9 is a partial vertical sectional view showing a coupling state of the light-emitting unit, the light-guiding unit and the support unit of the light-guiding type LED mask device according to one embodiment of the present disclosure. FIG. 10 is a perspective view of a light-guiding type light-emitting module of the light-guiding type LED mask device according to one embodiment of the present disclosure. FIG. 11 is a perspective view of a direct type light-emitting module of the light-guiding type LED mask device according to one embodiment of the present disclosure. FIG. 12 is a partial vertical sectional view showing a coupling state of the light-emitting unit, the light-guiding unit, the support unit and a reflection unit of the light-guiding type LED mask device according to one embodiment of the present disclosure. FIG. 13 is a partial vertical sectional view showing a coupling state of the light-emitting unit, the light-guiding unit, the support unit and the reflection unit of the light-guiding type LED mask device according to another embodiment of the present disclosure. FIG. 14 is a partial vertical sectional view showing a coupling state of the light-emitting unit, the light-guiding unit and the reflection unit of the light-guiding type LED mask device according to still another embodiment of the present disclosure. FIG. 15 is a partial vertical sectional view showing a coupling state of the light-emitting unit, the light-guiding unit and the reflection unit of the light-guiding type LED mask device according to still another embodiment of the present disclosure. FIG. 16 is a partial vertical sectional view showing a coupling state of the light-emitting unit, the light-guiding unit and the reflection unit of the light-guiding type LED mask device according to still another embodiment of the present disclosure. FIG. 17 is a schematic view illustrating a state in which light radiated from the light-emitting part of the light-guiding type LED mask device according to one embodiment is being dispersed by the light-guiding unit and the reflection unit. FIG. 18 is a schematic configuration view for a control unit of the light-guiding type LED mask device according to one embodiment of the present disclosure.

Referring to FIGS. 1 to 18, a light-guiding type LED mask device 100 according to one embodiment of the present disclosure includes a support unit 110, a light-emitting unit 120, a light-guiding unit 130, a reflection unit 140, and a control unit 170. Furthermore, the light-guiding type LED mask device 100 may further include a support cover unit 150 and a face-covering unit 160.

The light-guiding type LED mask device 100 is a device for irradiating light emitted from LED elements to a user's skin while being worn on the front of a user's face. In the light-guiding type LED mask device 100, the light-emitting unit 120 is placed at a location corresponding to an outer side of face with respect to the face, and the light is dispersed and radiated through the light-guiding unit 130, such that the light may be uniformly radiated to the entire face. Accordingly, in the light-guiding type LED mask device 100, an existing problem, that is, a phenomenon in which a relatively large amount of visible or near-infrared light is radiated to a skin in a region directly below the LED element, and relatively little visible or near-infrared light is radiated to the skin in a region farther from the LED element, does not occur. Meanwhile, the light-guiding type LED mask device utilizes visible light or near-infrared light, and may utilize both visible light and near-infrared light.

The support unit 110 includes a front opening 111, a rear insertion opening 112, and a head-covering region 113. In addition, the support unit 110 may include a plurality of element-coupling holes 114. The support unit 110 may be made of a synthetic resin material having a predetermined thickness. In addition, the support unit 110 may be formed of an opaque resin. On the whole, the support unit 110 is formed in a shape corresponding to the entire face and a portion of a front side of a head. The support unit 110 enables the light-guiding type LED mask device 100 to be worn on the face and a portion of the head. The control unit 170 may be supported on and coupled to the outside of the support unit 110.

The front opening 111 is formed by opening a region of the support unit 110 corresponding to an overall shape of the face. The front opening 111 provides a region to which the light-guiding part 130 is coupled. At an end portion of the front opening 111, a seating section 115 such as a seating jaw or a seating plate on which the light-emitting unit 120 is mounted may be positioned.

The rear insertion opening 112 is formed by opening a region of the support unit 110, which corresponds to the face and a portion of the front side of the head. The rear insertion opening 112 provides a path into which the face and the front side of the head are inserted when the light-guiding type LED mask device 100 is worn.

The head-covering region 113 is positioned at a location between an upper portion of the front opening 111 and an upper portion of the rear insertion opening 112 and corresponding to a portion of the front side of the head. The head-covering region 113 wraps a portion of the front side of the head.

The element-coupling hole 114 is formed to pass through upper and lower surfaces of the head-covering region 113. The plurality of element-coupling holes 114 are formed and may be spaced apart from each other to be dispersedly placed in the head-covering region 113.

The light-emitting unit 120 includes a light-guiding type light-emitting module 121. In addition, the light-emitting unit 120 may include a direct type light emitting-module 125. The light-emitting unit 120 generates visible light or near-infrared light.

The light-guiding type light emitting module 121 includes a light-guiding substrate 122 and a light-guiding LED element 123. The plurality of light-guiding type light emitting modules 121 are provide, and may be formed in a band shape. On the whole, the light-guiding type light-emitting module 121 may be formed in a linear shape or a curved shape. That is, the light-guiding type light-emitting module 121 may be formed in a straight shape 121a or a curved shape 121b according to a shape thereof attached to an outer peripheral surface of the front opening 111. The light-guiding type light-emitting module 121 is coupled to the outer peripheral surface of the front opening 111 of the support unit 110. The light-guiding type light-emitting module radiates visible light or near-infrared light to an outer surface of the light-guiding unit 130 coupled to the front opening 111. In addition, the light-guiding type light-emitting module 121 may radiate both visible light and near-infrared light.

The light-guiding substrate 122 is formed of a circuit board used in a conventional LED module. The light-guiding substrate 122 is formed in a band shape, and may also be formed in a linear shape or a curved shape. The light-guiding substrate 122 is coupled to the outer peripheral surface of the front opening 111 of the support unit 110.

The light-guiding LED elements 123 are mounted on the light-guiding substrate 122 to be spaced apart from each other at a predetermined interval. The light-guiding LED element 123 may be an element radiating visible light or near-infrared light, which have various wavelengths. In addition, the light-guiding LED element 123 may be an element radiating visible light or near-infrared light, which have a specific wavelength. The light-guiding LED element 123 may radiate visible light or near-infrared light in a direction perpendicular to the light-guiding substrate 122. Furthermore, the light-guiding LED element 123 may radiate both visible light and near-infrared light. The light-guiding LED element 123 may be composed of a LED having single wavelength or multiple wavelengths in a range of 400 to 900 nm.

The direct type light-emitting module 125 includes a direct substrate 126 and a direct LED element 127. The direct type light-emitting module 125 may be formed in a plate shape on the whole, and may be formed to have an area and a shape corresponding to those of the head-covering region 113. On the whole, the direct type light-emitting module 125 may be formed in a curved shape to correspond to the head-covering region 113. The direct type light-emitting module 125 is placed in the head-covering region 113 of the support unit 110, and may directly radiate light onto a surface of the head. For example, the direct type light-emitting module 125 may radiate light to the front side, the lateral side and the top of the head. In addition, the direct type light-emitting module 125 may radiate both visible light and near-infrared light.

The direct substrate 126 is formed of a circuit board used in a conventional LED module. The direct substrate 126 may be formed to have an area and a shape corresponding to those of the head-covering region 113.

The direct LED elements 127 are mounted to the direct substrate 126 to be spaced apart from each other at a predetermined interval. The direct LED element 127 may be placed at a position corresponding to the element-coupling hole 114. Also, the direct LED element 127 may be formed to be partially inserted into the element-coupling hole 114. The direct LED element 127 radiate light downward through the element-coupling hole 114. That is, the direct LED element 127 may directly radiate light to the surface of the head through the element-coupling hole 114.

The direct LED element 127 may be an element radiating visible light or near-infrared light, which have various wavelengths. In addition, the direct LED element 127 may be an element radiating visible light or near-infrared light with a specific wavelength. The direct LED element 127 may radiate both visible light and near-infrared light. The direct LED element 127 may be composed of a LED having single wavelength or multiple wavelengths in a range of 400 to 900 nm.

On the whole, the light-guiding unit 130 is formed in a shape corresponding to the face of the head. The light-guiding unit 130 may be formed of various materials employed for a light guide plate. The light-guiding unit 130 may be formed in a curved surface on the whole. The light-guiding unit 130 is formed such that a shape of an outer surface thereof corresponds to the front opening 111. The light-guiding unit 130 is coupled to the front opening 111. The light-guiding unit 130 is coupled such that an outer surface thereof faces an upper portion of the light-guiding LED element 123. In the light-guiding unit 130, light radiated from the light-guiding LED element 123 is incident thereon, and the incoming light is radiated to the rear surface. That is, light radiated from the light-guiding LED element 123 is incident on the outer surface of the light-guiding unit 130, and is reflected inside the light-guiding unit 130 and is then radiated to a rear surface.

The light-guiding unit 130 may include a light-guiding opening hole 131 formed at a position corresponding to an eye on the face. The light-guiding opening hole 131 may block light from being radiated to the face during use.

As shown in FIG. 12, the light-guiding unit 130 may be formed to have the same thickness on the whole. In addition, as shown in FIG. 13, the light-guiding unit 130 is formed to have a thickness which is reduced from an outer end to an inner side to a predetermined width. Furthermore, the light-guiding unit 130 may be formed such that a thickness of a region thereof corresponding to the front opening 111 has a uniform thickness on the whole. When a thickness of the light-guiding unit 130 is smaller than a size of the light-guiding LED element 123, an outer end thereof may be formed to be inclined.

Although not specifically illustrated, a rear surface of the light-guiding unit 130, that is, a surface facing the face may be coated with an antistatic agent. The antistatic agent may suppress generation of static electricity in the course of its action. The antistatic agent may be formed of a known material used for preventing static electrify on a plastic or resin material.

In addition, the antistatic agent may be dispersed and formed in the light-guiding unit 130. In this case, the antistatic agent may be mixed with a material of the light-guiding unit 130 before forming the light-guiding part 130.

The light-guiding unit 130 may be provided with a light-guiding pattern 132 formed on a front surface, a rear surface, or the front and rear surfaces, and having a shape of a groove or a protrusion. More specifically, if the light-guiding pattern 132 is formed in a shape of groove, it may be formed in a dot shape, or may extend in one direction to be formed in a trench shape on the whole. In addition, if the light-guiding pattern 132 is formed in a shape of protrusion, it may be formed in a dot shape or may extend in one direction to be formed in a wire shape on the whole.

Also, as shown in FIGS. 14 and 15, the light-guiding pattern 132 may be formed in a shape such as an inward concaved arc, semicircle, triangular, or square shape. In this case, a plurality of trench shapes are formed in the light-guiding pattern 132 to be spaced apart from each other.

The light-guiding pattern 132 may extend outward from a center of the light-guiding unit 130 to be formed as a radial-shaped pattern. In addition, a plurality of the light-guiding patterns 132 may be formed to be spaced apart from each other at a predetermined angle. The light-guiding pattern 132 may consist of 30 to 240 radial-shaped light-guiding patterns. Since the area of the light-guiding unit 130 is approximately defined with respect to a user's face, it is possible to adjust a distance between the light-guiding patterns by adjusting the number of radial-shaped light-guiding patterns. When the number of radial-shaped light-guiding patterns is small, the degree of increase in light dispersion is small, so light a light extraction degree may be reduced. On the other hand, if the number of radial-shaped light-guiding patterns is too large, the gap between the light-guiding patterns becomes narrow, which may cause manufacturing difficulties and increase manufacturing costs.

In addition, the light-guiding pattern 132 may be formed as a contour-shaped light-guiding pattern which forms a looped curve with respect to the center of the light-guiding unit 130. In this case, a plurality of the light-guiding patterns 132 may be formed to be spaced apart from each other by a predetermined distance. The light-guiding pattern 132 may consist of 30 to 240 contour-shaped light-guiding patterns. Since the area of the light-guiding unit 130 is approximately defined with respect to a user's face, it is possible to adjust a distance between the light-guiding patterns by adjusting the number of contour-shaped light-guiding patterns. When the number of contour-shaped light-guiding patterns is small, the degree of increase in light dispersion is small, so light a light extraction degree may be reduced. On the other hand, if the number of contour-shaped light-guiding patterns is too large, the gap between the light-guiding patterns becomes narrow, which may cause manufacturing difficulties and increase manufacturing costs.

In addition, the light-guiding pattern 132 may consist of the radial-shaped light-guiding pattern and the contour-shaped light-guiding pattern. In the light-guiding pattern 132, the radial-shaped light-guiding pattern and the contour-shaped light-guiding pattern may be formed together on a front surface of the light-guiding unit 130, that is, a surface corresponding a user's face, and a surface opposite thereto. Accordingly, the light-guiding pattern 132 may have a lattice shape on the whole. At this time, the radial-shaped light-guiding pattern may be formed to have a wire shape or a trench shape, and conversely, the contour-shaped light-guiding pattern may be formed to have a trench shape or a wire shape.

When both the radial-shaped light-guiding pattern and the contour-shaped light-guiding pattern are formed, the light-guiding pattern 132 may be preferably formed such that the number of radial-shaped light-guiding patterns is equal to or greater than the number of contour-shaped light-guiding patterns. When the radial-shaped light-guiding pattern is increased, light uniformity and a light extraction degree may be increased.

Also, as shown in FIG. 16, the light-guiding pattern 132 may be formed by distributing light-guiding particles inside the light-guiding unit 130. The light-guiding particles may be formed of a resin which is a material different from that of the light-guiding unit 130. For example, the light-guiding unit 130 may be formed of a resin material, and the light-guiding particles may be formed of inorganic particles. In addition, in the light-guiding unit 130, a polymer may be formed into the light-guiding particles by mixing and molding a transparent resin and a polymer in a liquid phase.

The reflection unit 140 is formed in a shape corresponding to the light-guiding unit 130 on the whole. On the whole, the reflective part 140 may be formed to have a curved surface. The reflection unit 140 is coupled such that a rear surface thereof comes into contact with the front surface of the light-guiding unit 130. Preferably, the reflection unit 140 is configured such that the rear surface of the reflection unit 140 may come into close contact with the front surface of the light-guiding unit 130. The reflection unit 140 reflects light traveling to the front surface of the light-guiding unit 130 again to allow light to enter the inside of the light-guiding unit 130. Accordingly, the reflection 140 increases the efficiency at which light radiated from the light-guiding type light-emitting module 121 reaches a user's face.

The reflection unit 140 may include a reflective opening hole 141 formed at a position corresponding to an eye of user's face. The reflective opening hole 141 may block light from being radiated to a user's face during use.

Referring to FIG. 17, the reflection unit 140 together with the light-guiding unit 130 allows light emitted from the light-guiding type light-emitting module 121 to be uniformly dispersed and radiated towards the rear surface of the light-guiding unit 130, that is, a user's face.

The support cover unit 150 is formed in a shape corresponding to the support unit 110 on the whole. The support cover unit 150 may be provided with a cover front opening 151, a cover rear insertion opening 152, and a cover head-covering region 153. The cover front opening 151, the cover rear insertion opening 152, and the cover head-covering region 153 of the support cover unit 150 may be formed to correspond to the front opening 111, the rear insertion opening, and the head-covering region 113 of the support unit 110, respectively.

The support cover unit 150 is coupled to wrap the light-emitting unit 120 coupled to the front opening 111 and the head-covering region 113 of the support unit 110. In addition, the support cover unit 150 may be coupled to cover outer surfaces of the light-guiding unit 130 and the reflection unit 140. Accordingly, the support cover part 150 prevents light radiated from the light-emitting unit 120 from leaking to the front surface. The support cover unit 150 may be formed of an opaque resin material.

The face-covering unit 160 is formed in a shape corresponding to the reflection part 140 on the whole. The face-covering unit 160 may be formed to have a curved surface on the whole. The face-covering unit 160 is coupled such that a rear surface thereof comes in contact with a front surface of the reflection unit 140. Preferably, the rear surface of the face cover unit 160 may come into close contact with the front surface of the reflection unit 140. The face-covering unit 160 is coupled to the cover front opening 151. Accordingly, the face-covering unit 160 may be coupled to wrap the front surface of the reflection unit 140 and an outer peripheral surface of the cover front opening 151. The face-covering unit 160 together with the support cover unit 150 prevents structures of the reflection unit 140 and the light-guiding unit 130 in the light-guiding type LED mask device 100 from being exposed to a front of the light-guiding type LED mask device. The face-covering unit 160 may be formed to be colored.

The control unit 170 controls the LED elements of the light-emitting unit 120. The control unit 170 may include a main control part 171, a light emission driving part 172, a manipulation part 173, a display part 174, a speaker 175, and a battery 176. The control unit 170 controls the light-guiding type light-emitting module 121 and the direct type light-emitting module 125 to emit light. In addition, the control unit 170 performs various operations necessary to control the light-guiding type light-emitting module 121 and the direct type light-emitting module 125. The control unit 170 may be embedded in the support unit 110 or the support cover unit 150, or may be formed as a separate module.

The main control part 171 controls the overall operation of the light-guiding type LED mask device 100.

The light emission driving part 172 supplies electricity to the light-guiding type light-emitting module 121 and the direct type light-emitting module to emit light from the light-emitting LED element 123 and the direct LED element 127. The light emission driving part 172 is driven with constant current, and may drive the LED elements simultaneously or sequentially to emit lights with various wavelengths.

The manipulation part 173 may include various buttons and switches necessary to operate the main control part 171 and the light emission driving part 172. For example, the manipulation part 173 may include an on-off switch for operating the main control part 171. In addition, the manipulation part 173 may be provided with an operation switch for driving the light emission driving part 172. In addition, the manipulation part 173 may select an operation for each wavelength in the light-guiding LED element or the direct LED element. The manipulation part 173 may use a proximity sensor to start light emission when worn and to finish light emission when detached.

The display part 174 may visually display an operation state of the main control part 171 or the light emission driving part 172. The display part 174 may be equipped with a general display device such as an LCD, an OLED, or an electronic display board, or a point light.

The speaker 175 may display an operation state of the main control part 171 or the light emission driving part 172 using voice or sound. The speaker 175 may be formed as a conventional speaker.

The battery 176 may supply electricity necessary for operation of the main control part 171 and the light emission driving part 172. In addition, the battery 176 may supply power to the light-guiding LED element 123 and the direct LED element 127. The battery 176 may be formed as a rechargeable battery capable of charging and discharging. The battery 176 may be charged by receiving an external power supply in a wire or wireless manner.

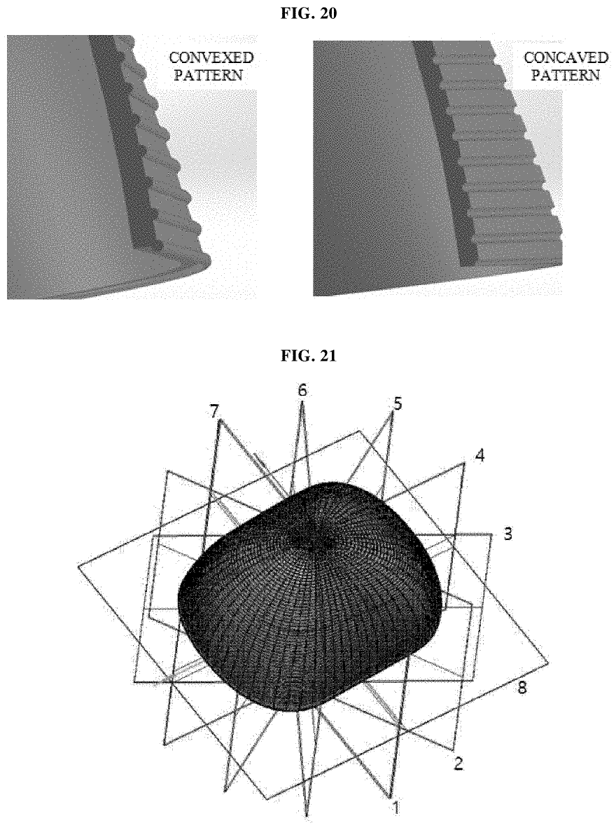

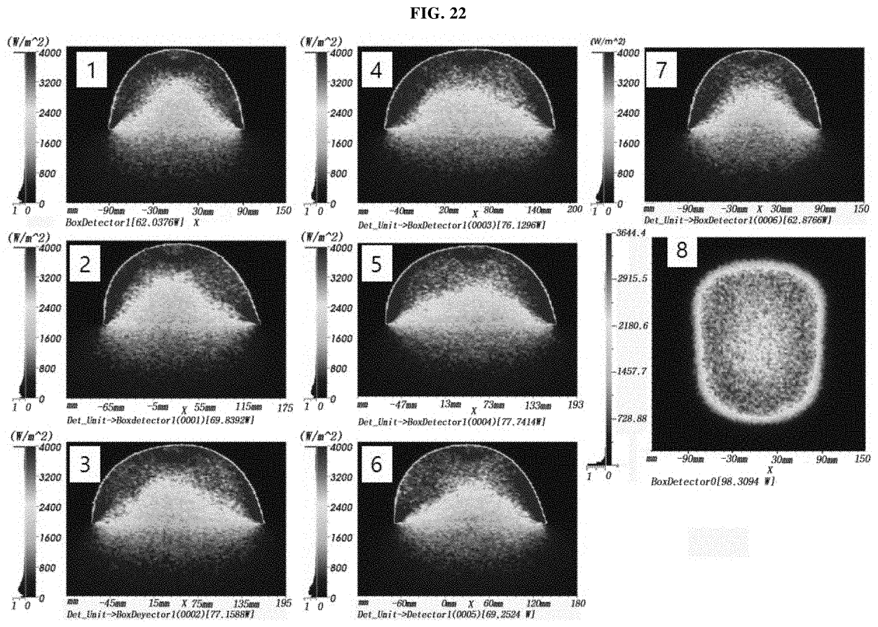

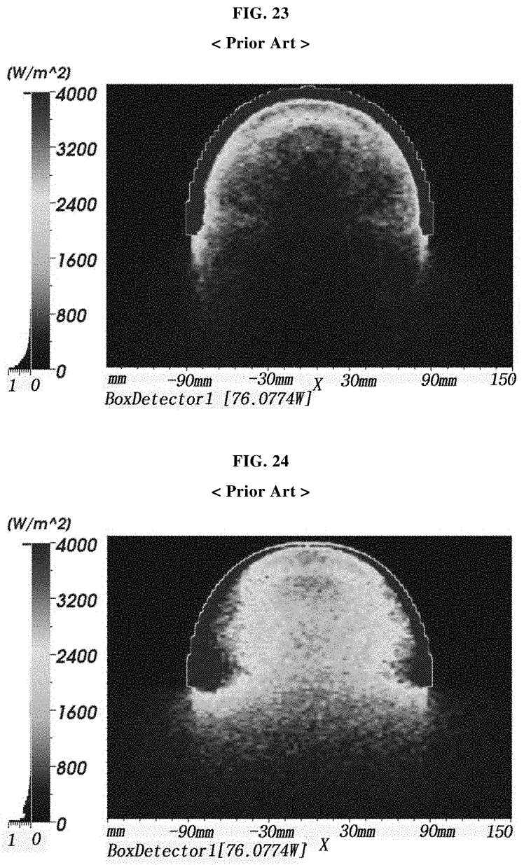

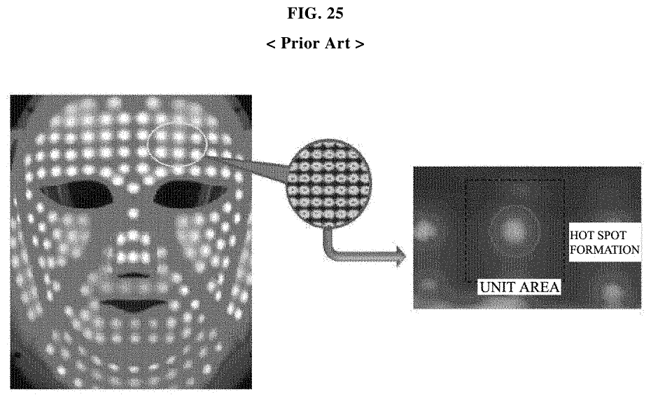

Next, a simulation result of the light-guiding type LED mask device 100 according to one embodiment of the present disclosure is described below. FIG. 19 is a view showing a mask model, a LED distribution and a pattern form for calculating a light extraction degree distribution of the light-guiding type LED mask device 100 according to one embodiment of the present disclosure. FIG. 20 is a view showing a specific form of a light-guiding pattern used in FIG. 19. FIG. 21 is a model picture illustrating locations at which the light extraction degree distribution is measured in the mask of FIG. 19. FIG. 22 is a graph for the light extraction degree distribution evaluated in eight directions of FIG. 21. FIG. 23 is a graph for the light extraction degree distribution evaluated in a state in which no light-guiding pattern is formed. FIG. 24 is a graph for luminous intensity distribution evaluated in a state in which a contour-shaped light-guiding pattern is formed and no radial-shaped light-guiding pattern is formed.

This simulation was conducted in order to fabricate the light-guiding unit 130 in the light-guiding type LED mask device 100, in which light is uniformly and entirely radiated to a user's face. This simulation evaluated the light extraction degree according to the light-guiding pattern to evaluate a light dispersion form coming out of the rear surface of the light-guiding unit 130. This simulation was conducted using an optical simulation program.

The light-guiding unit 130 was shaped using the 3D CAD to have a front mask type three-dimensional free curved surface. The light-guiding unit 130 was set to have a thickness of about 2 mm, a refractive index of 1.59, and 100 light-guiding type LED elements. In addition, the light-guiding unit 130 was set to emit light energy of 100 W from 100 LED elements. Furthermore, the light-guiding unit 130 was configured to reflect 100% of light from the front surface thereof.

Referring to FIG. 19, in the light-guiding unit 130, the radial-shaped light-guiding pattern and the contour-shaped light-guiding pattern were formed. The radial-shaped light-guiding patterns were formed to extend outward from the center of the light-guiding unit 130 and be spaced apart from each other at a predetermined angle. In addition, the contour-shaped light-guiding patterns were formed as contour-shaped light-guiding pattern forming looped curves with respect to the center of the light-guiding unit 130.

Referring to FIG. 20, the light-guiding pattern was formed as a convexed pattern or a concaved pattern. Here, the contour-shaped light-guiding pattern was formed as the convexed pattern, and the radial-shaped light-guiding pattern was formed as a concaved pattern. Also, in this simulation, the amount of light was detected while changing the number of light-guiding patterns. Here, 150 contour-shaped light-guiding patterns were formed and 240 radial-shaped light-guiding patterns were formed. In addition, the light-guiding patterns were formed on the front surface of the light-guiding unit 130, that is, on a surface opposite to the surface facing a user's face.

In this simulation, referring to FIG. 21, the amount of light was detected at first to eighths positions with respect to the center of the light-guiding unit 130.

Referring to FIG. 22, it can be seen that, in the light-guiding type LED mask device 100, light uniformity is generally high on a region where a user's face is located. In addition, the above-described light-guiding type LED mask device 100 tends to increase the light extraction degree to 98% or more.

In comparison, referring to FIG. 23, when no light-guiding pattern is formed on the light-guiding unit 130, the light extraction degree is low. In addition, even when the contour-shaped light-guiding pattern is formed on the front surface of the light-guiding unit 130 and no radial-shaped light-guiding pattern is not formed, referring to FIG. 24, the light extraction degree is relatively high in a peripheral region, and the light extraction degree is low in a central region.

In the above, although embodiments according to the technical idea of the present disclosure have been described with reference to the accompanying drawings, those of ordinary skill in the art to which the present disclosure pertains may understand that the present disclosure will be implemented as other specific forms without changing the technical spirit or essential features thereof. It should be understood that the embodiments described above are illustrative in all respects and not restrictive.

### INDUSTRIAL APPLICABILITY

The light-guiding type LED mask device according to one embodiment of the present disclosure is a device worn on an entire surface of a face to radiate light radiated from the LED element to a skin. In the above light-guiding type LED mask device, the light-emitting unit is placed at a position corresponding to the outside of the face with respect to the user's face, and light is distributed and radiated through the light-guiding unit, so that it is possible to uniformly radiate light to the entire face. Therefore, in the light-guiding type LED mask device, an existing problem, that is, a phenomenon in which a relatively large amount of visible or near-infrared light is radiated to a skin in a region directly below the LED element, and relatively little visible or near-infrared light is radiated to the skin in a region farther from the LED element, does not occur. Meanwhile, the light-guiding type LED mask device utilizes visible light or near-infrared light, and may utilize both visible light and near-infrared light.

1. A light-guiding type LED mask device, comprising:
a support unit formed in a shape corresponding to an entire face and a portion of a front side of a head on the whole, and being provided with a front opening and a rear insertion opening providing a path into which the face and the front side of the head are inserted;
a light-emitting unit configured to emit visible light or near-infrared light and provided with a light-guiding type light-emitting module attached to an outer periphery surface of the front opening;
a light-guiding unit coupled to the front opening and radiating light radiated from the light-guiding type light-emitting module; and
a reflection unit being in contact with a front surface of the light-guiding unit and configured to allow light, which travels to the front surface of the light-guiding unit, to enter the inside of the light-guiding unit.

2. The light-guiding type LED mask device of claim 1, wherein the support unit further comprises a head-covering region placed at a position corresponding to a portion of the front side of the head, and wherein the light-emitting unit is coupled to the head-cover region and further comprises a direct type light-emitting module configured to radiate visible light or near infrared light to a surface of the head.

3. The light-guiding type LED mask device of claim 1, wherein the light-guiding type light emitting module comprises a light-guiding substrate and a light-guiding LED element mounted to the light-guiding substrate, and wherein the light-guiding unit is coupled such that an outer surface thereof faces the light-guiding LED element, and is configured to radiate light radiated from the light-guiding LED element to a rear surface.

4. The light-guiding type LED mask device of claim 1, the light-guiding unit further comprises a light-guiding opening hole formed at a position corresponding to an eye on the face to block light from being radiated to the face.

5. The light-guiding type LED mask device of claim 1, wherein the light-guiding unit further comprises a light-guiding pattern.

6. The light-guiding type LED mask device of claim 5, wherein the light-guiding pattern is formed on a front surface, a rear surface, or the front and rear surfaces of the light-guiding unit and having a shape of a groove or a protrusion, and the light-guiding pattern comprises a radial-shaped light-guiding pattern extending outward from a center of the light-guiding unit.

7. The light-guiding type LED mask device of claim 6, wherein the light-guiding pattern is formed on a front surface, a rear surface, or the front and rear surfaces of the light-guiding unit and having a shape of a groove or a protrusion, and the light-guiding pattern comprises a contour-shaped light-guiding pattern which forms a looped curve with respect to a center of the light-guiding unit.

8. The light-guiding type LED mask device of claim 7, wherein the radial-shaped light-guiding pattern is formed to have a wire shape or a trench shape, and conversely, the contour-shaped light-guiding pattern is formed to have a trench shape or a wire shape.

9. The light-guiding type LED mask device of claim 1, wherein the light-guiding pattern is formed such that the number of radial-shaped light-guiding patterns is equal to or greater than the number of contour-shaped light-guiding patterns.

10. The light-guiding type LED mask device of claim 5, wherein the light-guiding pattern is formed by distributing light-guiding particles inside the light-guiding unit, and wherein the light guiding particle is formed of a resin which is a material different from that of the light-guiding unit.

11. A light-guiding type LED mask device, comprising:
a support unit formed in a shape corresponding to an entire face and a portion of a front side of a head on the whole, and being provided with a front opening and a rear insertion opening providing a path into which the face and the front side of the head are inserted;
a light-emitting unit provided with a light-guiding type light-emitting module attached to an outer periphery surface of the front opening;
a light-guiding unit coupled to the front opening and radiating light radiated from the light-guiding type light-emitting module, the light-guiding unit further comprises:
a light-guiding opening hole formed at a position corresponding to an eye on the face to block light from being radiated to the face; and
a light-guiding pattern; and
a reflection unit being in contact with a front surface of the light-guiding unit.

12. The light-guiding type LED mask device of claim 11, wherein the support unit further comprises a head-covering region placed at a position corresponding to a portion of the front side of the head, and wherein the light-emitting unit is coupled to the head-cover region and further comprises a direct type light-emitting module configured to radiate visible light or near infrared light to a surface of the head.

13. The light-guiding type LED mask device of claim 11, wherein the light-guiding type light emitting module comprises a light-guiding substrate and a light-guiding LED element mounted to the light-guiding substrate, and wherein the light-guiding unit is coupled such that an outer surface thereof faces the light-guiding LED element, and is configured to radiate light radiated from the light-guiding LED element to a rear surface.

14. The light-guiding type LED mask device of claim 11, wherein the light-guiding pattern is formed on a front surface, a rear surface, or the front and rear surfaces of the light-guiding unit and having a shape of a groove or a protrusion, and the light-guiding pattern comprises a radial-shaped light-guiding pattern extending outward from a center of the light-guiding unit.

15. The light-guiding type LED mask device of claim 13, wherein the light-guiding pattern is formed on a front surface, a rear surface, or the front and rear surfaces of the light-guiding unit and having a shape of a groove or a protrusion, and the light-guiding pattern comprises a contour-shaped light-guiding pattern which forms a looped curve with respect to a center of the light-guiding unit.

16. The light-guiding type LED mask device of claim 15, wherein the radial-shaped light-guiding pattern is formed to have a wire shape or a trench shape, and conversely, the contour-shaped light-guiding pattern is formed to have a trench shape or a wire shape.

17. The light-guiding type LED mask device of claim 11, wherein the light-guiding pattern is formed such that the number of radial-shaped light-guiding patterns is equal to or greater than the number of contour-shaped light-guiding patterns.

18. The light-guiding type LED mask device of claim 11, wherein the light-guiding pattern is formed by distributing light-guiding particles inside the light-guiding unit, and wherein the light guiding particle is formed of a resin which is a material different from that of the light-guiding unit.
